(19) United States
(12) Patent Application Publication
(10) Pub. No.: US 2009/0012178 A1
(43) Pub. Date: Jan. 8, 2009

(54) Benzoxazocines and Their Therapeutic Use

(75) Inventor: Andrew Douglas Baxter, Cambridge (GB)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: BEMIS MANFACTURING, SHEBOYGAN FALLS, WI (US)

(21) Appl. No.: 11/817,200

(22) PCT Filed: Mar. 10, 2006
(86) PCT No.: PCT/GB06/00858

§ 371 Date: Jun. 24, 2008

(30) Foreign Application Priority Data
Mar. 10, 2005 (GB) 0504960.6

(51) Int. Cl. A61K 31/166; C07D 267/14; A61P 13/00; A61P 25/00

(52) U.S. Cl. 514/622; 540/468

(57) ABSTRACT

A compound selected from (1S)-8-cyano-5-methyl-1-(3-methoxy)phenyl-1,3,4,6-tetrahydro-5H-benz[f]-2,5-oxazocine; (1R)-8-cyano-5-methyl-1-(3-methoxy)phenyl-1,3,4,6-tetrahydro-5H-benz[f]-2,5-oxazocine; (1S)-8-cyclopropyl-5-methyl-1-(3-methoxy)phenyl-1,3,4,6-tetrahydro-5H-benz[f]-2,5-oxazocine; (1S)-8-cyclopropyl-5-methyl-1-(3-methoxy)phenyl-1,3,4,6-tetrahydro-5H-benz[f]-2,5-oxazocine; (1S)-5-methyl-1-(3-methoxy)phenyl-1,3,4,6-tetrahydro-5H-benz[f]-2,5-oxazocine-8-carboxamide; (1R)-5-methyl-1-(3-methoxy)phenyl-1,3,4,6-tetrahydro-5H-benz[f]-2,5-oxazocine-8-carboxamide; and the salts thereof. These compounds have therapeutic utility.

### FIELD OF THE INVENTION

The present invention relates to novel benzoxazocines and to their therapeutic use, e.g. as analgesic agents.

### BACKGROUND OF THE INVENTION

Compounds based on the nefopam benzoxazocine template, having the ability to selectively inhibit the reuptake of the monoamines, noradrenaline (SNRIs) and serotonin (SSRIs), are described in WO2004/056788. In addition, this specification describes compounds that inhibit the uptake of both of these monoamines (S+NRIs). That specification is incorporated herein by reference. Compounds with improved potency and differentiated selectivity with respect to nefopam are described. Compounds such as 8-cyano-5-methyl-1-(3-methoxy)phenyl-1,3,4,6-tetrahydro-5H-benz[f]-2,5-oxazocine, 8-cyclopropyl-5-methyl-1-(3-methoxy)phenyl-1,3,4,6-tetrahydro-5H-benz[f]-2,5-oxazocine and 5-methyl-1-(3-methoxy)phenyl-1,3,4,6-tetrahydro-5H-benz[f]-2,5-oxazocine-8-carboxamide are disclosed as racemates.

Serotonin and noradrenaline are implicated in enhancing endogenous analgesic mechanisms via descending inhibitory pain pathways in the brain and spinal cord. In vivo microdialysis studies have shown that S+NRIs, such as duloxetine, are potent inhibitors of serotonin and noradrenaline reuptake in rodent brain. In addition, compounds such as duloxetine inhibit the late-stage paw licking behaviour in the formalin model of persistent pain in rats in a dose-related manner. In this respect, duloxetine has been shown to be more efficacious than venlafaxine, another S+NRI, and has a wider therapeutic window than amitriptyline where efficacy is only observed at doses which also lead to neuromuscular dysfunction in the rotorod test. SSRI's such as paroxetine and SNRI's such as thionisoxetine do not reverse formalin-induced late phase paw-licking behaviour when dosed alone but are efficacious when dosed in combination. These data support a role for both serotonin and noradrenaline being key mediators of descending pain pathways. Moreover, S+NRI by compounds such as duloxetine may offer a highly effective and safe treatment for persistent pain states in man.

In the clinical setting, SSRI's are known to suffer from dose-limiting side-effects, such as nausea, vomiting and sexual dysfunction, when used in the treatment of depression. SNRI's, such as atomoxetine, do not suffer the same side-effects. Duloxetine (a S+NRI) induces nausea in patients treated from depression, neuropathic pain and micturition disorders. In addition, the compound is both substantially metabolised by cytochrome P450 2D6 (CYP 2D6) and is an inhibitor of the same enzyme and is precluded for use in combination with both other CYP 2D6 inhibitors and compounds that are also substantially metabolised by CYP 2D6. This includes a number of clinically useful analgesic agents such as codeine and tramadol.

### SUMMARY OF THE INVENTION

The present invention is based on the observation that selected compounds display particularly advantageous properties when studied as their single enantiomers. For instance, the single enantiomer (1S)-(+)-8-cyano-5-methyl-1-(3-methoxy)phenyl-1,3,4,6-tetrahydro-5H-benz[f]-2,5-oxazocine is devoid of clinically relevant CYP 2D6 inhibitory activity (IC₅₀=5.7 μM) and is known to be metabolised by a range of cytochrome P450 enzymes. In addition, this enantiomer contains the majority of the SSRI activity (IC₅₀=8.9 nM) of the racemic mixture and displays a greatly improved pharmacokinetic profile over the parent compound nefopam. Furthermore, unlike duloxetine, (1S)-(+)-8-cyano-5-methyl-1-(3-methoxy)phenyl-1,3,4,6-tetrahydro-5H-benz[f]-2,5-oxazocine reverses both early and late-phase formalin-induced paw licking in rats, indicating that this compound may find utility in both acute and persistent pain. This is supported by the compound's efficacy in acute pain models such as the mouse hot-plate model where the (1S)-(+)-enantiomer has greater efficacy than the racemate, which in turn has greater efficacy than the (1R)-(−)-enantiomer.

Unexpectedly, the situation is slightly different for 8-cyclopropyl-5-methyl-1-(3-methoxy)phenyl-1,3,4,6-tetrahydro-5H-benz[f]-2,5-oxazocine. In this case, both enantiomers have interesting but quite distinct biological activity. (1S)-(+)-cyclopropyl-5-methyl-1-(3-methoxy)phenyl-1,3,4,6-tetrahydro-5H-benz[f]-2,5-oxazocine is a potent S+NRI (IC₅₀=5.2 nM as SRI and 46 nM as NRI), but this compound also has some CYP 2D6 inhibition (IC₅₀=1.1 μM). In contrast, (1R)-(−)-cyclopropyl-5-methyl-1-(3-methoxy)phenyl-1,3,4,6-tetrahydro-5H-benz[f]-2,5-oxazocine has a more SSRI profile (IC₅₀=21 nM as SRI and >1 μM as NRI) and demonstrates a greatly reduced CYP 2D6 inhibition (IC₅₀=10 μM). Both enantiomers may find utility in the treatment of both acute and persistent pain. Indeed, unlike 8-cyano-5-methyl-1-(3-methoxy)phenyl-1,3,4,6-tetrahydro-5H-benz[f]-2,5-oxazocine, both enantiomers of cyclopropyl-5-methyl-1-(3-methoxy)phenyl-1,3,4,6-tetrahydro-5H-benz[f]-2,5-oxazocine are active in the mouse hot-plate model.

In addition, both (1S)-(+)-cyclopropyl-5-methyl-1-(3-methoxy)phenyl-1,3,4,6-tetrahydro-5H-benz[f]-2,5-oxazocine and (1S)-(+)-cyano-5-methyl-1-(3-methoxy)phenyl-1,3,4,6-tetrahydro-5H-benz[f]-2,5-oxazocine have been shown to be anti-emetic in the morphine-induced emesis model in ferrets. The implication is that these single enantiomers have the potential to be analgesic but devoid of the nausea and emesis associated with other compounds of this class.

### DESCRIPTION OF THE INVENTION

Compounds of the invention are single isomers. This means that they are in at least 80%, preferably at least 90%, more preferably at least 95% and most preferably at least 99%, enantomeric excess with respect to the opposite isomer.

The present invention relates to the use of single enantiomers of selected compounds, pharmaceutically active salts, polymorphs and ‘active’ metabolites, their pharmaceutical formulations and their therapeutic utility as analgesics. In addition, compounds of this class are useful in a wide range of other indications including, but not limited to, depression, post-traumatic stress disorders, substance abuse, addiction, alcohol or drug dependence (e.g. nicotine addiction, alcoholism); akathisia (restless legs syndrome); anxiety/depression disorders (e.g. bipolar disorder, general anxiety disorder, major depressive disorder, mood disorder, social anxiety disorder, panic disorder, premenstrual disorder, obsessive-compulsive disorders, post-traumatic stress disorder), attention deficit hyperactivity disorder; central nervous system diseases (e.g. Alzheimer's disease, dementia, dyskinesia, neurodegenerative disorders, Parkinson's disease, schizophrenia), eating disorders (e.g. anorexia, binge eating, bulimia), fatigue-related syndromes (e.g. fibromyalgia syndrome, chronic fatigue syndrome, complex regional pain syndrome, myofacial pain and atypical chest pain), functional bowel disorders (e.g. Irritable bowel syndrome), menopause, micturition disorders (e.g. stress urinary incontinence, urge incontinence), migraine, obesity, pain (e.g. acute, chronic benign pain or neuropathic pain including diabetic neuropathy and post-herpetic neuralgia, post-operative pain, pain associated with cancer, surgery, arthritis, dental surgery, painful neuropathies, trauma, musculo-skeletal injury or disease, visceral diseases, dysmenorrhea or migraine headache), peripheral vascular disease, pruritus; sleep disorder, sexual dysfunction (e.g. erectile dysfunction, female sexual dysfunction, premature ejaculation)

For the treatment of pain, and in other diseases and indications such as those highlighted above, the claimed compounds may be administered orally, topically, parenterally, by inhalation or nasal spray or rectally in dosage unit formulations containing non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats etc, the compounds of the invention may be effective in the treatment of humans.

A pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyeryl distearate may be employed. They may also be coated by the techniques described in U.S. Pat. No. 4,256,108, U.S. Pat. No. 4,166,452 and U.S. Pat. No. 4,265,874 to form osmotic therapeutic tablets for control release.

Formulations for oral use may also be presented as hard gelatin capsules where in the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc containing the compounds are employed. For purposes of this specification, topical application includes mouth washes and gargles.

Dosage levels of the order of from about 0.05 mg to about 140 mg per kilogram of body weight per day are useful in the treatment of the above-indicated conditions (about 2.5 mg to about 7 g per patient per day). For example, pain may be effectively treated by the administration of from about 0.01 to 50 mg of the compound per kilogram of body weight per day (about 0.5 mg to about 3.5 g per patient per day).

The amount of active ingredient that may be combined with the carrier materials to produce a single dosage form will vary depending upon the host treated and the particular mode of administration. For example, a formulation intended for the oral administration of humans may vary from about 5 to about 95 percent of the total composition. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

Single enantiomers of the invention may be separated from the corresponding racemate by chiral HPLC.

The following Examples illustrate the invention.

### EXAMPLE 1

### (1S)-8-cyano-5-methyl-1-(3-methoxy)phenyl-1,3,4,6-tetrahydro-5H-benz[f]-2,5-oxazocine

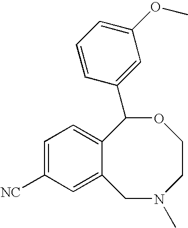

Racemic material was prepared as described in WO2004/056788, Example 22. 8-Cyano-5-methyl-1-(3-methoxy)phenyl-1,3,4,6-tetrahydro-5H-benz[f]-2,5-oxazocine (3 g) was purified on a CHIRALPAK AD 20 g/m (250 mm×50 mm) chromatography column, with an eluent of 100% acetonitrile, a flow rate of 120 ml/min and UV wavelength detection at 290 nm. 1.02 g of a yellow oil was isolated as the second eluting peak, HPLC 98.8%, enantiomeric excess>99.5.

### EXAMPLE 2

### (1R)-8-cyano-5-methyl-1-(3-methoxy)phenyl-1,3,4,6-tetrahydro-5H-benz[f]-2,5-oxazocine

Racemic material was prepared as described in WO2004/056788, Example 22. 8-Cyano-5-methyl-1-(3-methoxy)phenyl-1,3,4,6-tetrahydro-5H-benz[f]-2,5-oxazocine (3 g) was purified on a CHIRALPAK AD 20 μm (250 mm×50 mm) chromatography column, with an eluent of 100% acetonitrile, a flow rate of 120 ml/min and UV wavelength detection at 290 nm. 528 mg of a brown solid was isolated as the first eluting peak, HPLC 97.0%, enantiomeric excess>99.5.

### EXAMPLE 3

### Single Enantiomer 8-cyclopropyl-5-methyl-1-(3-methoxy)phenyl-1,3,4,6-tetrahydro-5H-benz[f]-2,5-oxazocine (Second Eluting Peak)

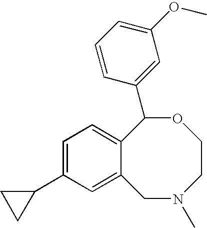

Racemic material was prepared as described in WO2004/056788, Example 29. 8-Cyclopropyl-5-methyl-1-(3-methoxy)phenyl-1,3,4,6-tetrahydro-5H-benz[f]-2,5-oxazocine (300 mg) was purified on a CHIRALPAK AD 20 μm (250 mm×10 mm) chromatography column, with an eluent of 100% methanol, a flow rate of 9 ml/min and UV wavelength detection at 280 nm. 93 mg of a pink viscous oil was isolated as the second eluting peak, HPLC 97.0%, enantiomeric excess>99.5.

### EXAMPLE 4

### Single Enantiomer 8-cyclopropyl-5-methyl-1-(3-methoxy)phenyl-1,3,4,6-tetrahydro-5H-benz[f]-2,5-oxazocine (First Eluting Peak)

Racemic material was prepared as described in WO2004/056788, Example 29. 8-Cyclopropyl-5-methyl-1-(3-methoxy)phenyl-1,3,4,6-tetrahydro-5H-benz[f]-2,5-oxazocine (300 mg) was purified on a CHIRALPAK AD 20 μm (250 mm×10 mm) chromatography column, with an eluent of 100% methanol, a flow rate of 9 ml/min and UV wavelength detection at 280 nm. 89 mg of a pink viscous oil was isolated as the first eluting peak, HPLC 97.2%, enantiomeric excess>99.5.

### EXAMPLE 5

### Single Enantiomer 5-methyl-1-(3-methoxy)phenyl-1,3,4,6-tetrahydro-5H-benz[f]-2,5-oxazocine-8-carboxamide (Second Eluting Peak)

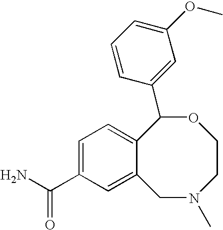

Racemic material was prepared as described in WO05103019, Example 10. 5-Methyl-1-(3-methoxy)phenyl-1,3,4,6-tetrahydro-5H-benz[f]-2,5-oxazocine-8-carboxamide (105 mg) was purified on a CHIRALPAK AD 20 μm (245 mm×50 mm) chromatography column, with an eluent of 100% methanol, a flow rate of 120 ml/min and UV wavelength detection at 250 nm. 50 mg of a clear glass was isolated as the second eluting peak., HPLC 98.1%, enantiomeric excess 96.3.

### EXAMPLE 6

### Single Enantiomer 5-methyl-1-(3-methoxy)phenyl-1,3,4,6-tetrahydro-5H-benz[f]-2,5-oxazocine-8-carboxamide (First Eluting Peak)

Racemic material was prepared as described in WO05103019, Example 10. 5-Methyl-1-(3-methoxy)phenyl-1,3,4,6-tetrahydro-5H-benz[f]-2,5-oxazocine-8-carboxamide (105 mg) was purified on a CHIRALPAK AD 20 μm (245 mm×50 mm) chromatography column, with an eluent of 100% methanol, a flow rate of 120 ml/min and UV wavelength detection at 250 nm. 50 mg of a clear glass was isolated as the first eluting peak, HPLC 99.6%, enantiomeric excess 99.4.

The following studies provide evidence on which the present invention is based.

### CYP 2D6 Inhibition

Compounds of the invention have been shown to have differential activity against the serotonin and noradrenaline transporters and have been tested in a substrate based assay to determine inhibition against CYP 2D6. CYP 2D6 inhibition assays were conducted according to the method of Ono et al. (1996—Xenobiotica, 26: 681-693). 5-HT Transporter binding assays were conducted according to the method of Tatsumi et al. (1997—Eur. J. Pharmacol., 340: 249-258). NA Transporter binding assays were conducted according to the method of Pacholczyk et al. (1991—Nature, 350: 350-354). Results are given in Table 1.

TABLE 1

| Assay | Example 1 | Racemate | Example 2 |
|---|---|---|---|
| IC₅₀ vs SRI (nM) | 8.9 | 19 | 120 |
| IC₅₀ vs NRI (nM) | 400 | 1100 | 3800 |
| IC₅₀ vs CYP2D6 (nM) | 5700 | 7100 | 9600 |
| Assay | Example 3 | Racemate | Example 4 |
| IC₅₀ vs SRI (nM) | 5.2 | 8.3 | 21 |
| IC₅₀ vs NRI (nM) | 46 | 46 | >1000 |
| IC₅₀ vs CYP2D6 (nM) | 1100 | 2000 | >10000 |

### Mouse Hotplate Model of Acute Analgesia

Compounds of the invention have also been evaluated in the mouse hotplate model of acute analgesia. Mice placed on a metallic hot plate will respond by paw licking or by jumping up from the plate (Eddy et al., 1950—J. Pharmacol. Exp. Ther.; 98:121-137). Analgesics increase nociceptive reaction latency.

Mice (17-23 g male Swiss mice ICO: OF1) were placed on a metallic hot plate maintained at 56±0.2° C. The nociceptive reaction latency, characterized by a licking reflex of the forepaws or by a jumping off the hot plate, was recorded. Plate heat cut-off time was 30 seconds. The test substances and vehicle were orally administered 60 min before testing. Results are given in Table 2 (nt=not tested; *denotes statistical significance achieved).

TABLE 2

| Paw Latency (Sec) | | | |
|---|---|---|---|
| above control | Example 1 | Racemate | Example 2 |
| 10 mg/kg po | 9* | nt | nt |
| 30 mg/kg po | 5 | nt | 6 |
| 60 mg/kg po | 7* | 8* | 1 |
| 100 mg/kg po | nt | 6 | nt |
| Paw Latency (Sec) | | | |
| above control | Example 3 | Racemate | Example 4 |
| 10 mg/kg po | 3 | nt | nt |
| 30 mg/kg po | 6* | 5 | 5 |
| 60 mg/kg po | 9* | 11* | 5 |
| 100 mg/kg po | nt | nt | nt |

### Formalin-Induced Paw Licking in Mouse

Compounds of the invention have also been evaluated in the formalin-induced paw licking model in mice. Compounds were evaluated for both an early stage response and a late stage response against duloxetine and nefopam as controls.

Inflammation was induced by subplantar injection of a 5% formalin solution (0.02 ml) into the mouse right hindpaw (20-25 g male Rj: NMRI). Hindpaw licking time was continuously recorded in a blinded fashion between 0 to 5 minutes (early phase) and between 20 to 30 minutes (late phase) after formalin injection (Hunskaar et al., 1985—J. Neurosci. Methods; 14:69-76).

The test substances and vehicle were orally administered 60 min before formalin injection. Results are given in Table 3 (nt=not tested; *denotes statistical significance achieved).

TABLE 3

| | | | | |
|---|---|---|---|---|
| Nociception | Nefopam | Example 1 | Example 3 | Duloxetine |

Early Phase:

| | | | | |
|---|---|---|---|---|
| 30 mg/kg po | nt | −49% | −45% | −49% |
| 60 mg/kg po | −46% | −75%* | −56%* | nt |
| 100 mg/kg po | −76%* | nt | nt | nt |

Late Phase:

| | | | | |
|---|---|---|---|---|
| 30 mg/kg po | nt | −46% | −51% | −85%* |
| 60 mg/kg po | −26% | −88%* | −73%* | nt |
| 100 mg/kg po | −68%* | nt | nt | nt |

### Morphine Challenge Assay

Compounds of the invention have been demonstrated to be anti-emetic in ferrets following a morphine challenge. In this respect, compounds of the invention retain the low side-effect potential of nefopam while having greater efficacy and more favourable pharmacokinetic parameters for chronic use.

Morphine was administered (0.125 mg/Kg s.c.) to albino or fitch male ferrets (0.9-1.7 kg) to induce emesis. Emesis was characterized by rhythmic abdominal contractions which were either associated with the oral expulsion of solid or liquid material from the gastrointestinal tract (ie vomiting) or not associated with the passage of material (i.e. retching movements). The number of highly distinctive abdominal contractions was counted.

The test substances and vehicle were administered by intraperitoneal injection 60 min before morphine administration. Results are given in Table 4 (*denotes statistical significance achieved).

TABLE 4

| % Reduction in no | | | |
|---|---|---|---|
| of retches/vomits | Example 1 | Nefopam | Example 3 |
| 3 mg/kg ip | 91%* | 96%* | 89%* |

### Behavioural Despair Test

Compounds of the invention have been evaluated in the Behavioural Despair Test, a model which detects antidepressant activity.

The Behavioural Despair Test was conducted according to the method of Porsolt et al., (1977—Arch. Int. Pharmacodyn., 229:327-336). Mice forced to swim in a situation from which they cannot escape rapidly become immobile. Antidepressants decrease the duration of immobility.

Mice (20-27 g male Rj: NMRI) were individually placed in a cylinder (height=24 cm, diameter=13 cm) containing 10 cm water (22° C.), from which they cannot escape. The mice were placed in the water for 6 minutes and the duration of immobility during the last 4 minutes was measured. All compounds were administered i.p. 30 minutes before the test, and compared with a vehicle control group. Results are given in Table 5 (nt=not tested; *denotes statistical significance achieved).

TABLE 5

| Duration of Immobility (% | | | |
|---|---|---|---|
| change from ctrl) | Example 1 | Duloxetine | Imipramine |
| 10 mg/kg ip | −24* | −6 | nt |
| 20 mg/kg ip | −8 | −9 | nt |
| 32 mg/kg ip | nt | nt | −62* |
| 40 mg/kg ip | −61* | −65* | nt |
| 80 mg/kg ip | −85* | −100* | nt |

1. A compound selected from
(1S)-8-cyano-5-methyl-1-(3-methoxy)phenyl-1,3,4,6-tetrahydro-5H-benz[f]-2,5-oxazocine;
(1R)-8-cyano-5-methyl-1-(3-methoxy)phenyl-1,3,4,6-tetrahydro-5H-benz[f]-2,5-oxazocine;
(1S)-8-cyclopropyl-5-methyl-1-(3-methoxy)phenyl-1,3,4,6-tetrahydro-5H-benz[f]-2,5-oxazocine;
(1S)-8-cyclopropyl-5-methyl-1-(3-methoxy)phenyl-1,3,4,6-tetrahydro-5H-benz[f]-2,5-oxazocine;
(1S)-5-methyl-1-(3-methoxy)phenyl-1,3,4,6-tetrahydro-5H-benz[f]-2,5-oxazocine-8-carboxamide;
(1R)-5-methyl-1-(3-methoxy)phenyl-1,3,4,6-tetrahydro-5H-benz[f]-2,5-oxazocine-8-carboxamide; and
the salts thereof.

2. The compound according to claim 1, which is (1S)-8-cyano-5-methyl-1-(3-methoxy)phenyl-1,3,4,6-tetrahydro-5H-benz[f]-2,5-oxazocine.

3. The compound according to claim 1, which is (1R)-8-cyano-5-methyl-1-(3-methoxy)phenyl-1,3,4,6-tetrahydro-5H-benz[f]-2,5-oxazocine.

4. The compound according to claim 1, which is (1S)-8-cyclopropyl-5-methyl-1-(3-methoxy)phenyl-1,3,4,6-tetrahydro-5H-benz[f]-2,5-oxazocine.

5. The compound according to claim 1, which is (1S)-8-cyclopropyl-5-methyl-1-(3-methoxy)phenyl-1,3,4,6-tetrahydro-5H-benz[f]-2,5-oxazocine.

6. The compound according to claim 1, which is (1S)-5-methyl-1-(3-methoxy)phenyl-1,3,4,6-tetrahydro-5H-benz[f]-2,5-oxazocine-8-carboxamide.

7. The compound according to claim 1, which is (1R)-5-methyl-1-(3-methoxy)phenyl-1,3,4,6-tetrahydro-5H-benz[f]-2,5-oxazocine-8-carboxamide.

8. A pharmaceutical composition for use in therapy, comprising a compound according to claim 1, and a pharmaceutically acceptable diluent or carrier.

9. A method for the treatment or prevention of a condition associated with monoamine re-uptake wherein said method comprises administering, to a patient in need of such treatment or prevention, a compound selected from
(1S)-8-cyano-5-methyl-1-(3-methoxy)phenyl-1,3,4,6-tetrahydro-5H-benz[f]-2,5-oxazocine;
(1R)-8-cyano-5-methyl-1-(3-methoxy)phenyl-1,3,4,6-tetrahydro-5H-benz[f]-2,5-oxazocine;
(1S)-8-cyclopropyl-5-methyl-1-(3-methoxy)phenyl-1,3,4,6-tetrahydro-5H-benz[f]-2,5-oxazocine;
(1S)-8-cyclopropyl-5-methyl-1-(3-methoxy)phenyl-1,3,4,6-tetrahydro-5H-benz[f]-2,5-oxazocine;
(1S)-5-methyl-1-(3-methoxy)phenyl-1,3,4,6-tetrahydro-5H-benz[f]-2,5-oxazocine-8-carboxamide;
(1R)-5-methyl-1-(3-methoxy)phenyl-1,3,4,6-tetrahydro-5H-benz[f]-2,5-oxazocine-8-carboxamide; and
the salts thereof.

10. The method according to claim 9, where the condition is acute, chronic or neuropathic pain, dysmennorhoea or migraine headache.

11. The method according to claim 10, wherein the subject is also treated with an opiate.

12. The method according to claim 10, wherein the subject is also treated with an analgesia inducer selected from acetaminophen, a non-steroidal anti-inflammatory drug, a narcotic analgesic, a local anesthetic, an NMDA antagonist, a neuroleptic agent, an anti-convulsant, an anti-spasmodic, an anti-depressant or a muscle relaxant.

13. The method according to claim 9, wherein the condition is emesis.

14. The method according to claim 13, wherein the emesis is acute, delayed, post-operative, last-phase or anticipatory emesis.

15. The method according to claim 13, wherein the emesis is induced by chemotherapy, radiation, toxins, pregnancy, vestibular disorder, motion, post-operative sickness, surgery, gastrointestinal obstruction, reduced gastrointestinal motility, visceral pain, migraine or opioid analgesics.

16. The method according to claim 9, wherein the condition is substance abuse, addiction or alcohol or drug dependence.

17. The method according to claim 9, wherein the condition is akathisia, depression, or an anxiety/depression disorder.

18. The method according to claim 9, wherein the condition is an attention-deficit hyperactivity disorder.

19. The method according to claim 9, wherein the condition is a central nervous system disease.

20. The method according to claim 9, wherein the condition is an eating disorder.

21. The method according to claim 9, wherein the condition is a fatigue-related syndrome.

22. The method according to claim 9, wherein the condition is a functional bowel disorder.

23. The method according to claim 9, wherein the condition is selected from menopause, obesity, peripheral vascular disease, pruritus, sleep disorder and sexual dysfunction.

24. The method according to claim 9, wherein the condition is a micturition disorder such as stress urinary incontinence or urge incontinence.
